(19) United States
(12) Patent Application Publication
(10) Pub. No.: US 2022/0040080 A1
(43) Pub. Date: Feb. 10, 2022

(54) PERSONAL CARE COMPOSITION

(71) Applicants: Rohm and Haas Company, Collegeville, PA (US); Union Carbide Corporation, Seadrift, TX (US)

(72) Inventors: Lyndsay M. Leal, Spring City, PA (US); Emmett M. Partain, III, Bound Brook, NJ (US); Nikhil J. Fernandes, Philadelphia, PA (US); Daniel S. Miller, Phoenixville, PA (US)

(21) Appl. No.: 17/276,336

(22) PCT Filed: Nov. 1, 2019
(86) PCT No.: PCT/US2019/059343

§ 371 Date: Mar. 15, 2021

Related U.S. Application Data
(60) Provisional application No. 62/767,653, filed on Nov. 15, 2018.

(51) Int. Cl. A61K 8/73; A61K 8/46; A61K 8/42; A61K 8/44; A61K 8/37; A61K 8/898; A61K 8/891; A61Q 5/02; A61Q 5/12

(52) U.S. Cl. CPC A61K 8/731; A61K 8/463; A61K 8/42; A61K 8/44; A61K 8/37; A61K 8/898; A61K 2800/596; A61Q 5/02; A61Q 5/12; A61K 2800/48; A61K 2800/51; A61K 2800/524; A61K 2800/594; A61K 8/891

(57) ABSTRACT

A personal care composition is provided, comprising: a cosmetically acceptable carrier; a cosmetically acceptable silicone; a modified carbohydrate polymer; wherein the modified carbohydrate polymer comprises a cellulose ether base material functionalized with (i) quaternary ammonium groups, wherein the quaternary ammonium groups and wherein the modified carbohydrate polymer has a Kjeldahl nitrogen content corrected for ash and volatiles, TKN, of 1.0 to 4.0 wt %; and (ii) hydrophobic substituents, wherein the hydrophobic substituents comprise an alkyl group having an average of 10 to 20 carbon atoms; wherein the modified carbohydrate polymer comprises >0.005 to <0.05 moles of the hydrophobic substituents per mole of the cellulose ether base material; wherein the hydrophobic groups are randomly distributed across the backbone of the cellulose ether base material; and wherein the cellulose ether base material has a weight average molecular weight, Mw, of >200,000 Daltons.

The present invention relates to a personal care composition. In particular, the present invention relates to a personal care composition containing: a cosmetically acceptable carrier; a cosmetically acceptable silicone; a modified carbohydrate polymer; wherein the modified carbohydrate polymer comprises a cellulose ether base material functionalized with (i) quaternary ammonium groups, wherein the quaternary ammonium groups and wherein the modified carbohydrate polymer has a Kjeldahl nitrogen content corrected for ash and volatiles, TKN, of 1.0 to 4.0 wt %; and (ii) hydrophobic substituents, wherein the hydrophobic substituents comprise an alkyl group having an average of 10 to 20 carbon atoms; wherein the modified carbohydrate polymer comprises >0.005 to <0.05 moles of the hydrophobic substituents per mole of the cellulose ether base material; wherein the hydrophobic groups are randomly distributed across the backbone of the cellulose ether base material; and wherein the cellulose ether base material has a weight average molecular weight, Mw, of >200,000 Daltons.

Deposition of silicone is of particular interest for various personal care compositions. In particular, there is interest for personal care cleansers (e.g., body wash, face wash, hand wash, soap, shampoo, shampoo conditioners and hair conditioners) that provide moisturizing/conditioning benefits in addition to cleaning benefits.

Hair cleansing has become an ubiquitous component of personal hygiene. Cleansing of the hair facilitates the removal of dirt, germs and other things that are perceived as harmful to the hair or the individual. Cleansing formulations typically including a surfactant to promote the removal of materials deposited on the hair. Unfortunately, the cleansing formulations remove both undesirable and desirable materials from hair. For example, cleansing formulations frequently undesirably remove oils from hair, which oils operate to protect hair from loss of moisture. Removal of too much oil from hair may leave the hair vulnerable to becoming dry and damaged. One solution to this concern is the selection of mild surfactants. Another approach is to incorporate additives that help replace the oils removed through deposition; however, this approach has proven difficult in implementation, particularly in rinse off applications.

In U.S. Pat. No. 7,067,499, Erazo-Majewicz, et al disclose personal care and household care product composition comprising at least one cationic polygalactomannan or a derivative of cationic polygalactomannans wherein the derivative moiety on the cationic derivatized polygalactomannan is selected from the group consisting of alkyl, hydroxyalkyl, alkylhydroxyalkyl, and carboxymethyl wherein the alkyl has a carbon chain containing from 1 to 22 carbons and the hydroxyalkyl is selected from the group consisting of hydroxyethyl, hydroxypropyl, and hydroxybutyl, wherein the at least one cationic polygalactomannan or derivative of cationic polygalactomannans have a mean average molecular weight (Mw) having a lower limit of 5,000 and an upper limit of 200,000 and having a light transmittance in a 10% aqueous solution of greater than 80% at a light wavelength of 600 nm and a protein content of less than 1.0% by weight of polysaccharide, and aldehyde functionality content of at least 0.01 meg/gram.

While conventionally used deposition aids such as soluble cationic modified celluloses (e.g., polyquaternium-10), guar hydroxypropyltriammonium chloride and other cationic polymers (e.g., polyquaternium-6, polyquaternium-7) provide a certain level of deposition in personal care cleansers; they nevertheless exhibit low efficiency necessitating a relatively high incorporation of the active into the personal care cleanser formulation to facilitate desired results. Such high active (e.g., silicone) levels, however, detrimentally effect the foam/lathery in use consumer feel of the formulation and cost.

Accordingly, there remains a need for deposition aids that facilitate enhanced efficiency of silicone deposition from personal care compositions.

The present invention provides a personal care composition, comprising: a cosmetically acceptable carrier; a cosmetically acceptable silicone; a modified carbohydrate polymer; wherein the modified carbohydrate polymer comprises a cellulose ether base material functionalized with (i) quaternary ammonium groups, wherein the modified carbohydrate polymer has a Kjeldahl nitrogen content corrected for ash and volatiles, TKN, of 1.0 to 4.0 wt %; and (ii) hydrophobic substituents, wherein the hydrophobic substituents comprise an alkyl group having an average of 10 to 20 carbon atoms; wherein the modified carbohydrate polymer comprises >0.005 to <0.05 moles of the hydrophobic substituents per mole of the cellulose ether base material; and wherein the hydrophobic groups are randomly distributed across the backbone of the cellulose ether base material; and wherein the cellulose ether base material has a weight average molecular weight, Mw, of >200,000 Daltons.

The present invention provides a method of depositing silicone, comprising: selecting a personal care composition of the present invention; and applying the personal care composition to at least one of skin and hair.

### DETAILED DESCRIPTION

We have surprisingly found that silicone deposition from personal care formulations can be enhanced through incorporation of a modified carbohydrate polymer comprising a cellulose ether base material functionalized with quaternary ammonium groups and hydrophobic substituents; wherein the cellulose ether base material has a weight average molecular weight, MW, of >200,000 Daltons; wherein the modified carbohydrate polymer has a Kjeldahl nitrogen content corrected for ash and volatiles, TKN, of 1.0 to 4.0 wt %; wherein the modified carbohydrate polymer comprises >0.005 to <0.05 moles of the hydrophobic substituents per mole of the cellulose ether base material; and wherein the hydrophobic groups are randomly distributed across the backbone of the cellulose ether base material.

Unless otherwise indicated, ratios, percentages, parts, and the like are by weight.

As used herein, unless otherwise indicated, the phrase “molecular weight” or Mw refers to the weight average molecular weight as measured in a conventional manner with gel permeation chromatography (GPC) and conventional standards, such as polyethylene glycol standards. GPC techniques are discussed in detail in Modern Size Exclusion Chromatography, W. W. Yau, J. J. Kirkland, D. D. Bly; Wiley-Interscience, 1979, and in A Guide to Materials Characterization and Chemical Analysis, J. P. Sibilia; VCH, 1988, p. 81-84. Molecular weights are reported herein in units of Daltons, or equivalently, g/mol.

The term “cosmetically acceptable” as used herein and in the appended claims refers to ingredients typically used in personal care compositions, and is intended to underscore that materials that are toxic when present in the amounts typically found in personal care compositions are not contemplated as part of the present invention.

Preferably, the personal care composition of the present invention is selected from the group consisting of a body wash formulation, an exfoliating body wash formulation, a facial wash formulation, an exfoliating facial wash formulation, a liquid hand soap, a shampoo, a conditioning shampoo, a leave on hair conditioner, a rinse off hair conditioner, a hair coloring agent, a hair styling gel, a soap, a sunscreen, a sulfate-free cleansing formulation and a mild cleansing formulation. More preferably, the personal care composition of the present invention is selected from the group consisting of a shampoo, a conditioning shampoo, a leave on hair conditioner and a rinse off hair conditioner. Most preferably, the personal care composition of the present invention is a shampoo, a conditioner or a conditioning shampoo.

Preferably, the personal care composition of the present invention, comprises: a cosmetically acceptable carrier (preferably, wherein the personal care composition comprises 25 to 99 wt % (more preferably, 30 to 97.5 wt %; still more preferably, 60 to 95 wt %; most preferably, 75 to 90 wt %), based on weight of the personal care composition, of the cosmetically acceptable carrier (preferably, wherein the cosmetically acceptable carrier comprises water; more preferably; wherein the cosmetically acceptable carrier is selected from the group consisting of water and an aqueous C₁₋₄ alcohol mixture; most preferably, wherein the cosmetically acceptable carrier is water)); a cosmetically acceptable silicone (preferably, wherein the personal care composition comprises 0.1 to 10 wt % (more preferably, 0.1 to 5 wt %; still more preferably, 0.4 to 2.5 wt %; most preferably, 0.5 to 1.5 wt %), based on weight of the personal care composition, of the cosmetically acceptable silicone; a modified carbohydrate polymer (preferably, wherein the personal care composition comprises 0.1 to 10 wt % (more preferably, 0.15 to 2 wt %; still more preferably, 0.2 to 1 wt %; most preferably, 0.25 to 0.5 wt %), based on weight of the personal care composition, of the modified carbohydrate polymer); wherein the modified carbohydrate polymer comprises a cellulose ether base material functionalized with (i) quaternary ammonium groups, wherein the quaternary ammonium groups and wherein the modified carbohydrate polymer has a Kjeldahl nitrogen content corrected for ash and volatiles, TKN, of 1.0 to 4.0 wt % (preferably, 1.5 to 3.25; more preferably, 1.6 to 2.6; most preferably, 1.7 to 2.1); and (ii) hydrophobic substituents, wherein the hydrophobic substituents comprise an alkyl group having an average of 10 to 20 carbon atoms (preferably, 12 to 18 carbon atoms; more preferably, 12 to 16 carbon atoms; most preferably, 12 carbon atoms); wherein the modified carbohydrate polymer comprises >0.005 to <0.05 moles (preferably, 0.0075 to 0.045 moles; more preferably, 0.008 to 0.0425 moles; still more preferably, 0.009 to 0.04 moles; most preferably, 0.01 to 0.02 moles) of the hydrophobic substituents per mole of the cellulose ether base material; and wherein the hydrophobic groups are randomly distributed across the backbone of the cellulose ether base material; and wherein the cellulose ether base material has a weight average molecular weight, Mw, of >200,000 Daltons (preferable, >500,000 to 3,000,000 Daltons; more preferably, 600,000 to 2,500,000 Daltons; still more preferably, >800,000 to 2,250,000 Daltons; most preferably, 900,000 to 2,000,000 Daltons).

Preferably, the personal care composition of the present invention, comprises a cosmetically acceptable carrier. More preferably, the personal care composition of the present invention, comprises: 25 to 99 wt % (preferably, 30 to 97.5 wt %; more preferably, 60 to 95 wt %; most preferably, 75 to 90 wt %), based on weight of the personal care composition, of a cosmetically acceptable carrier. Still more preferably, the personal care composition of the present invention, comprises: 25 to 99 wt % (preferably, 30 to 97.5 wt %; more preferably, 60 to 95 wt %; most preferably, 75 to 90 wt %), based on weight of the personal care composition, of a cosmetically acceptable carrier; wherein the cosmetically acceptable carrier comprises water. Yet more preferably, the personal care composition of the present invention, comprises: 25 to 99 wt % (preferably, 30 to 97.5 wt %; more preferably, 60 to 95 wt %; most preferably, 75 to 90 wt %), based on weight of the personal care composition, of a cosmetically acceptable carrier; wherein the cosmetically acceptable carrier is selected from the group consisting of water and an aqueous C₁₋₄ alcohol mixture. Most preferably, the personal care composition of the present invention, comprises: 25 to 99 wt % (preferably, 30 to 97.5 wt %; more preferably, 60 to 95 wt %; most preferably, 75 to 90 wt %), based on weight of the personal care composition, of a cosmetically acceptable carrier; wherein the cosmetically acceptable carrier is water.

Preferably, the water used in the personal care composition of the present invention is at least one of distilled water and deionized water. More preferably, the water used in the personal care composition of the present invention is distilled and deionized.

Preferably, the personal care composition of the present invention comprises a cosmetically acceptable silicone. More preferably, the personal care composition of the present invention comprises 0.1 to 10 wt % (preferably, 0.1 to 10 wt %; more preferably, 0.1 to 5 wt %; still more preferably, 0.4 to 2.5 wt %; most preferably, 0.5 to 1.5 wt %), based on weight of the personal care composition, of a cosmetically acceptable silicone (preferably, wherein the cosmetically acceptable silicone conditions hair). Still more preferably, the personal care composition of the present invention comprises 0.1 to 10 wt % (preferably, 0.1 to 10 wt %; more preferably, 0.1 to 5 wt %; still more preferably, 0.4 to 2.5 wt %; most preferably, 0.5 to 1.5 wt %), based on weight of the personal care composition, of a cosmetically acceptable silicone, wherein the cosmetically acceptable silicone is selected from the group consisting of amodimethicone, cyclomethicone, dimethicone, dimethiconol, hexadecyl methicone, hexamethyldisiloxane, methicone, phenyl dimethicone, stearoxy dimethicone polyalkyl siloxane, polyalkylaryl siloxane, silicone gums (i.e., polydiorganosiloxanes having a weight average molecular weight of 200,000 to 1,000,000 Daltons), polyaminofunctional silicones (e.g., Dow Corning® 929) and mixtures thereof. Yet more preferably, the personal care composition of the present invention comprises 0.1 to 10 wt % (preferably, 0.1 to 10 wt %; more preferably, 0.1 to 5 wt %; still more preferably, 0.4 to 2.5 wt %; most preferably, 0.5 to 1.5 wt %), based on weight of the personal care composition, of a cosmetically acceptable silicone, wherein the cosmetically acceptable silicone is selected from the group consisting of amodimethicone, cyclomethicone, dimethicone, dimethiconol, hexadecyl methicone, hexamethyldisiloxane, methicone, phenyl dimethicone, stearoxy dimethicone and mixtures thereof. Still yet more preferably, the personal care composition of the present invention comprises 0.1 to 10 wt % (preferably, 0.1 to 10 wt %; more preferably, 0.1 to 5 wt %; still more preferably, 0.4 to 2.5 wt %; most preferably, 0.5 to 1.5 wt %), based on weight of the personal care composition, of a cosmetically acceptable silicone, wherein the cosmetically acceptable silicone is selected from the group consisting of amodimethicone, cyclomethicone, dimethicone, dimethiconol, hexadecyl methicone, methicone and mixtures thereof. Yet still more preferably, the personal care composition of the present invention comprises 0.1 to 10 wt % (preferably, 0.1 to 10 wt %; more preferably, 0.1 to 5 wt %; still more preferably, 0.4 to 2.5 wt %; most preferably, 0.5 to 1.5 wt %), based on weight of the personal care composition, of a cosmetically acceptable silicone, wherein the cosmetically acceptable silicone is selected from the group consisting of amodimethicone, dimethicone, dimethiconol and a mixture thereof. Most preferably, the personal care composition of the present invention comprises 0.1 to 10 wt % (preferably, 0.1 to 10 wt %; more preferably, 0.1 to 5 wt %; still more preferably, 0.4 to 2.5 wt %; most preferably, 0.5 to 1.5 wt %), based on weight of the personal care composition, of a cosmetically acceptable silicone, wherein the cosmetically acceptable silicone comprises a dimethiconol.

Preferably, the personal care composition of the present invention comprises a modified carbohydrate polymer. More preferably, the personal care composition of the present invention comprises 0.1 to 10 wt % (preferably, 0.15 to 2 wt %; more preferably, 0.2 to 1 wt %; most preferably, 0.25 to 0.5 wt %), based on weight of the personal care composition, of a modified carbohydrate polymer. Most preferably, the personal care composition of the present invention comprises 0.1 to 10 wt % (preferably, 0.15 to 2 wt %; more preferably, 0.2 to 1 wt %; most preferably, 0.25 to 0.5 wt %), based on weight of the personal care composition, of a modified carbohydrate polymer; wherein the modified carbohydrate polymer comprises a cellulose ether base material functionalized with (i) quaternary ammonium groups, wherein the quaternary ammonium groups and wherein the modified carbohydrate polymer has a Kjeldahl nitrogen content corrected for ash and volatiles, TKN, of 1.0 to 4.0 wt % (preferably, 1.5 to 3.25; more preferably, 1.6 to 2.6; most preferably, 1.7 to 2.1); and (ii) hydrophobic substituents, wherein the hydrophobic substituents comprise an alkyl group having an average of 10 to 20 carbon atoms (preferably, 12 to 18 carbon atoms; more preferably, 12 to 16 carbon atoms; most preferably, 12 carbon atoms); wherein the modified carbohydrate polymer comprises >0.005 to <0.05 moles (preferably, 0.0075 to 0.045 moles; more preferably, 0.008 to 0.0425 moles; still more preferably, 0.009 to 0.04 moles; most preferably, 0.01 to 0.02 moles) of the hydrophobic substituents per mole of the cellulose ether base material; and wherein the hydrophobic groups are randomly distributed across the backbone of the cellulose ether base material; and wherein the cellulose ether base material has a weight average molecular weight, Mw, of >200,000 Daltons (preferable, >500,000 to 3,000,000 Daltons; more preferably, 600,000 to 2,500,000 Daltons; still more preferably, >800,000 to 2,250,000 Daltons; most preferably, 900,000 to 2,000,000 Daltons).

Preferably, the cellulose ether base material has a weight average molecular weight, Mw, of >200,000 Daltons (preferable, >500,000 to 3,000,000 Daltons; more preferably, 600,000 to 2,500,000 Daltons; still more preferably, >800,000 to 2,250,000 Daltons; most preferably, 900,000 to 2,000,000 Daltons). More preferably, the cellulose ether base material has a weight average molecular weight, Mw, of >200,000 Daltons (preferable, >500,000 to 3,000,000 Daltons; more preferably, 600,000 to 2,500,000 Daltons; still more preferably, >800,000 to 2,250,000 Daltons; most preferably, 900,000 to 2,000,000 Daltons); wherein the cellulose ether base material is selected from the group consisting of hydroxyethyl cellulose, hydroxypropyl cellulose, ethyl hydroxyethyl cellulose, methyl cellulose, hydroxypropyl methyl cellulose, hydroxyethyl methyl cellulose and mixtures thereof. Still more preferably, the cellulose ether base material has a weight average molecular weight, Mw, of >200,000 Daltons (preferable, >500,000 to 3,000,000 Daltons; more preferably, 600,000 to 2,500,000 Daltons; still more preferably, >800,000 to 2,250,000 Daltons; most preferably, 900,000 to 2,000,000 Daltons); wherein the cellulose ether base material is selected from the group consisting of hydroxyethyl cellulose, hydroxypropyl cellulose and mixtures thereof. Most preferably, the cellulose ether base material has a weight average molecular weight, Mw, of >200,000 Daltons (preferable, >500,000 to 3,000,000 Daltons; more preferably, 600,000 to 2,500,000 Daltons; still more preferably, >800,000 to 2,250,000 Daltons; most preferably, 900,000 to 2,000,000 Daltons); wherein the cellulose ether base material is hydroxyethyl cellulose.

Preferably, the personal care composition of the present invention comprises 0.1 to 10 wt % (preferably, 0.15 to 2 wt %; more preferably, 0.2 to 1 wt %; most preferably, 0.25 to 0.5 wt %), based on weight of the personal care composition, of a modified carbohydrate polymer; wherein the modified carbohydrate polymer comprises a cellulose ether base material functionalized with (i) quaternary ammonium groups, wherein the modified carbohydrate polymer has a Kjeldahl nitrogen content corrected for ash and volatiles, TKN, of 1.0 to 4.0 wt % (preferably, 1.5 to 3.25; more preferably, 1.6 to 2.6; most preferably, 1.7 to 2.1); and wherein the quaternary ammonium groups are selected from the group consisting of trialkyl ammonium moieties having formula (A)

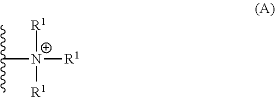

wherein each R¹ is independently selected from the group consisting of a C₁₋₃ alkyl group (preferably, a methyl group and an ethyl group; most preferably, a methyl group). More preferably, the personal care composition of the present invention comprises 0.1 to 10 wt % (preferably, 0.15 to 2 wt %; more preferably, 0.2 to 1 wt %; most preferably, 0.25 to 0.5 wt %), based on weight of the personal care composition, of a modified carbohydrate polymer; wherein the modified carbohydrate polymer comprises a cellulose ether base material functionalized with (i) quaternary ammonium groups, wherein the modified carbohydrate polymer has a Kjeldahl nitrogen content corrected for ash and volatiles, TKN, of 1.0 to 4.0 wt % (preferably, 1.5 to 3.25; more preferably, 1.6 to 2.6; most preferably, 1.7 to 2.1); and wherein the quaternary ammonium groups are trimethyl ammonium moieties. Most preferably, the personal care composition of the present invention comprises 0.1 to 10 wt % (preferably, 0.15 to 2 wt %; more preferably, 0.2 to 1 wt %; most preferably, 0.25 to 0.5 wt %), based on weight of the personal care composition, of a modified carbohydrate polymer; wherein the modified carbohydrate polymer comprises a cellulose ether base material functionalized with (i) quaternary ammonium groups, wherein the modified carbohydrate polymer has a Kjeldahl nitrogen content corrected for ash and volatiles, TKN, of 1.0 to 4.0 wt % (preferably, 1.5 to 3.25; more preferably, 1.6 to 2.6; most preferably, 1.7 to 2.1); wherein the quaternary ammonium groups are trimethyl ammonium moieties; and wherein the modified carbohydrate polymer comprises <0.1 moles (preferably, <0.01 moles; more preferably, <0.001 moles; most preferably, less than a detectable limit) of trialkyl ammonium moieties having formal (B) per mole of the cellulose ether base material

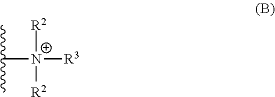

wherein each R² is independently selected from a methyl group and an ethyl group and wherein R³ is selected from a C₆₋₂₂ alkyl group.

Preferably, the personal care composition of the present invention, comprises 0.1 to 10 wt % (preferably, 0.15 to 2 wt %; more preferably, 0.2 to 1 wt %; most preferably, 0.25 to 0.5 wt %), based on weight of the personal care composition, of a modified carbohydrate polymer; wherein the modified carbohydrate polymer comprises a cellulose ether base material functionalized with (ii) hydrophobic substituents, wherein the hydrophobic substituents comprise an alkyl group having an average of 10 to 20 carbon atoms (preferably, 12 to 18 carbon atoms; more preferably, 12 to 16 carbon atoms; most preferably, 12 carbon atoms); wherein the modified carbohydrate polymer comprises >0.005 to <0.05 moles (preferably, 0.0075 to 0.045 moles; more preferably, 0.008 to 0.0425 moles; still more preferably, 0.009 to 0.04 moles; most preferably, 0.01 to 0.02 moles) of the hydrophobic substituents per mole of the cellulose ether base material; and wherein the hydrophobic groups are randomly distributed across the backbone of the cellulose ether base material. More preferably, the personal care composition of the present invention, comprises 0.1 to 10 wt % (preferably, 0.15 to 2 wt %; more preferably, 0.2 to 1 wt %; most preferably, 0.25 to 0.5 wt %), based on weight of the personal care composition, of a modified carbohydrate polymer; wherein the modified carbohydrate polymer comprises a cellulose ether base material functionalized with (ii) hydrophobic substituents, wherein the hydrophobic substituents comprise an alkyl group having an average of 10 to 20 carbon atoms (preferably, 12 to 18 carbon atoms; more preferably, 12 to 16 carbon atoms; most preferably, 12 carbon atoms) bonded to the cellulose ether base material through at least one of an ether linkage (e.g., an ether linkage alone or an ether linkage and a 2-hydroxypropyl group), an ester linkage, an amide linkage and a urethane linkage; wherein the modified carbohydrate polymer comprises >0.005 to <0.05 moles (preferably, 0.0075 to 0.045 moles; more preferably, 0.008 to 0.0425 moles; still more preferably, 0.009 to 0.04 moles; most preferably, 0.01 to 0.02 moles) of the hydrophobic substituents per mole of the cellulose ether base material; and wherein the hydrophobic groups are randomly distributed across the backbone of the cellulose ether base material. Still more preferably, the personal care composition of the present invention, comprises 0.1 to 10 wt % (preferably, 0.15 to 2 wt %; more preferably, 0.2 to 1 wt %; most preferably, 0.25 to 0.5 wt %), based on weight of the personal care composition, of a modified carbohydrate polymer; wherein the modified carbohydrate polymer comprises a cellulose ether base material functionalized with (ii) hydrophobic substituents, wherein the hydrophobic substituents comprise an alkyl group having an average of 10 to 20 carbon atoms (preferably, 12 to 18 carbon atoms; more preferably, 12 to 16 carbon atoms; most preferably, 12 carbon atoms) bonded to the water-soluble cellulose ether base material through at least one of an ether linkage (e.g., an ether linkage alone or an ether linkage and a 2-hydroxypropyl group), an ester linkage, an amide linkage and a urethane linkage; wherein the modified carbohydrate polymer comprises >0.005 to <0.05 moles (preferably, 0.0075 to 0.045 moles; more preferably, 0.008 to 0.0425 moles; still more preferably, 0.009 to 0.04 moles; most preferably, 0.01 to 0.02 moles) of the hydrophobic substituents per mole of the cellulose ether base material; and wherein the hydrophobic groups are randomly distributed across the backbone of the cellulose ether base material. Most preferably, the personal care composition of the present invention, comprises 0.1 to 10 wt % (preferably, 0.15 to 2 wt %; more preferably, 0.2 to 1 wt %; most preferably, 0.25 to 0.5 wt %), based on weight of the personal care composition, of a modified carbohydrate polymer; wherein the modified carbohydrate polymer comprises a cellulose ether base material functionalized with (ii) hydrophobic substituents, wherein the hydrophobic substituents comprise an alkyl group having an average of 10 to 20 carbon atoms (preferably, 12 to 18 carbon atoms; more preferably, 12 to 16 carbon atoms; most preferably, 12 carbon atoms) bonded to the water-soluble cellulose ether base material via an ether linkage alone (as in Formula I) or an ether linkage and a 2-hydroxypropyl group (as in Formula II)

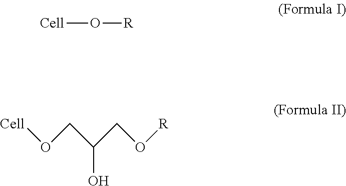

wherein Cell-O is the water-soluble cellulose ether base material and wherein R is the C₁₀₋₂₀
(preferably, C₁₂₋₁₈; more preferably, C₁₂₋₁₆; most preferably, C₁₂) linear or branched alkyl group; wherein the modified carbohydrate polymer comprises >0.005 to <0.05 moles (preferably, 0.0075 to 0.045 moles; more preferably, 0.008 to 0.0425 moles; still more preferably, 0.009 to 0.04 moles; most preferably, 0.01 to 0.02 moles) of the hydrophobic substituents per mole of the cellulose ether base material; and wherein the hydrophobic groups are randomly distributed across the backbone of the cellulose ether base material.

Preferably, the personal care composition of the present invention, optionally, further comprises at least one additional ingredient selected from the group consisting of a personal care cleansing surfactant; an antimicrobial agent/preservative (e.g., benzoic acid, sorbic acid, phenoxyethanol, methylisothiazolinone); a rheology modifier (e.g., PEG-150 pentaerythrityl tetrastearate); a soap; a colorant; pH adjusting agent; an antioxidant (e.g., butylated hydroxytoluene); a humectant (e.g., glycerin, sorbitol, monoglycerides, lecithins, glycolipids, fatty alcohols, fatty acids, polysaccharides, sorbitan esters, polysorbates (e.g., Polysorbate 20, Polysorbate 40, Polysorbate 60, and Polysorbate 80), diols (e.g., propylene glycol), diol analogs, triols, triol analogs, cationic polymeric polyols); a wax; a foaming agent; an emulsifying agent; a colorant; a fragrance; a chelating agent (e.g., tetrasodium ethylene diamine tetraacetic acid); a preservative (e.g., benzoic acid, sorbic acid, phenoxyethanol, methylisothiazolinone); a bleaching agent; a lubricating agent; a sensory modifier; a sunscreen additive; a vitamin; a protein/amino acid; a plant extract; a natural ingredient; a bioactive agent; an anti-aging agent; a pigment; an acid; a penetrant; an anti-static agent; an anti-frizz agent; an antidandruff agent; a hair waving/straightening agent; a hair styling agent; a hair oil; an absorbent; a hard particle; a soft particle; a conditioning agent (e.g., guar hydroxypropyltrimonium chloride, PQ-10, PQ-7); a slip agent; an opacifier; a pearlizing agent and a salt. More preferably, the personal care composition of the present invention, optionally, further comprises at least one additional ingredient selected from the group consisting of a personal care cleansing surfactant; an antimicrobial agent/preservative (e.g., benzoic acid, sorbic acid, phenoxyethanol, methylisothiazolinone); a rheology modifier (e.g., PEG-150 pentaerythrityl tetrastearate); and a chelating agent (e.g., tetrasodium ethylene diamine tetraacetic acid). Most preferably, the personal care composition of the present invention, optionally, further comprises at least one additional ingredient selected from the group consisting of a personal care cleansing surfactant; an antimicrobial agent/preservative mixture of phenoxyethanol and methylisothiazolinone; PEG-150 pentaerythrityl tetrastearate; tetrasodium ethylene diamine tetraacetic acid and a mixture of phenoxyethanol and methylisothiazolinone.

Preferably, the personal care composition of the present invention further comprises a personal care cleaning surfactant. More preferably, the personal care composition of the present invention further comprises a personal care cleansing surfactant, wherein the personal care cleansing surfactant is selected from the group consisting of alkyl polyglucosides (e.g., lauryl glucoside, coco-glucoside, decyl glucoside), glycinates (e.g., sodium cocoyl glycinate), betaines (e.g., alkyl betaines such as cetyl betaine and amido betaines such as cocamidopropyl betaine), taurates (e.g., sodium methyl cocoyl taurate), glutamates (e.g., sodium cocoyl glutamate), sarcosinates (e.g., sodium lauroyl sarcosinate), isethionates (e.g., sodium cocoyl isethionate, sodium lauroyl methyl isethionate), sulfoacetates (e.g., sodium lauryl sulfoacetate), alaninates (e.g., sodium cocoyl alaninate), amphoacetates (e.g., sodium cocoamphoacetate), sulfates (e.g., sodium lauryl ether sulfate (SLES)), sulfonates (e.g., sodium C₁₄₋₁₆ olefin sulfonate), succinates (e.g., disodium lauryl sulfosuccinate), fatty alkanolamides (e.g., cocamide monoethanolamine, cocamide diethanolamine, soyamide diethanolamine, lauramide diethanolamine, oleamide monoisopropanolamine, stearamide monoethanolamine, myristamide monoethanolamine, lauramide monoethanolamine, capramide diethanolamine, ricinoleamide diethanolamine, myristamide diethanolamine, stearamide diethanolamine, oleylamide diethanolamine, tallowamide diethanolamine, lauramide monoisopropanolamine, tallowamide monoethanolamine, isostearamide diethanolamine, isostearamide monoethanolamine) and mixtures thereof. Still more preferably, the personal care composition of the present invention further comprises a personal care cleaning surfactant; wherein the personal care composition is selected from the group consisting of a shampoo and a conditioning shampoo; and wherein the personal care cleaning surfactant is selected from the group consisting of alkyl polyglucosides (e.g., lauryl glucoside, coco-glucoside, decyl glucoside), glycinates (e.g., sodium cocoyl glycinate), betaines (e.g., alkyl betaines such as cetyl betaine and amido betaines such as cocamidopropyl betaine), taurates (e.g., sodium methyl cocoyl taurate), glutamates (e.g., sodium cocoyl glutamate), sarcosinates (e.g., sodium lauroyl sarcosinate), isethionates (e.g., sodium cocoyl isethionate, sodium lauroyl methyl isethionate), sulfoacetates (e.g., sodium lauryl sulfoacetate), alaninates (e.g., sodium cocoyl alaninate), amphoacetates (e.g., sodium cocoamphoacetate), sulfates (e.g., sodium lauryl ether sulfate (SLES)), sulfonates (e.g., sodium C₁₄₋₁₆ olefin sulfonate), succinates (e.g., disodium lauryl sulfosuccinate), fatty alkanolamides (e.g., cocamide monoethanolamine, cocamide diethanolamine, soyamide diethanolamine, lauramide diethanolamine, oleamide monoisopropanolamine, stearamide monoethanolamine, myristamide monoethanolamine, lauramide monoethanolamine, capramide diethanolamine, ricinoleamide diethanolamine, myristamide diethanolamine, stearamide diethanolamine, oleylamide diethanolamine, tallowamide diethanolamine, lauramide monoisopropanolamine, tallowamide monoethanolamine, isostearamide diethanolamine, isostearamide monoethanolamine) and mixtures thereof. Most preferably, the personal care composition of the present invention further comprises a personal care cleaning surfactant; wherein the personal care composition is selected from the group consisting of a shampoo and a conditioning shampoo; and wherein the personal care cleaning surfactant comprises a mixture of a betaine (preferably, cocamidopropyl betaine), a sulfate (preferably, sodium lauryl ether sulfate (SLES)), and a fatty alkanolamide (preferably, cocamide monoethanolamine)

Preferably, the personal care composition of the present invention further comprises 0.01 to 80 wt % (more preferably, 1 to 50 wt %; still more preferably, 5 to 35 wt %, most preferably, 7.5 to 20 wt %) of a personal care cleaning surfactant. More preferably, the personal care composition of the present invention further comprises 0.01 to 80 wt % (more preferably, 1 to 50 wt %; still more preferably, 5 to 35 wt %, most preferably, 7.5 to 20 wt %) of a personal care cleaning surfactant, wherein the personal care cleaning surfactant is selected from the group consisting of alkyl polyglucosides (e.g., lauryl glucoside, coco-glucoside, decyl glucoside), glycinates (e.g., sodium cocoyl glycinate), betaines (e.g., alkyl betaines such as cetyl betaine and amido betaines such as cocamidopropyl betaine), taurates (e.g., sodium methyl cocoyl taurate), glutamates (e.g., sodium cocoyl glutamate), sarcosinates (e.g., sodium lauroyl sarcosinate), isethionates (e.g., sodium cocoyl isethionate, sodium lauroyl methyl isethionate), sulfoacetates (e.g., sodium lauryl sulfoacetate), alaninates (e.g., sodium cocoyl alaninate), amphoacetates (e.g., sodium cocoamphoacetate), sulfates (e.g., sodium lauryl ether sulfate (SLES)), sulfonates (e.g., sodium C₁₄₋₁₆ olefin sulfonate), succinates (e.g., disodium lauryl sulfosuccinate), fatty alkanolamides (e.g., cocamide monoethanolamine, cocamide diethanolamine, soyamide diethanolamine, lauramide diethanolamine, oleamide monoisopropanolamine, stearamide monoethanolamine, myristamide monoethanolamine, lauramide monoethanolamine, capramide diethanolamine, ricinoleamide diethanolamine, myristamide diethanolamine, stearamide diethanolamine, oleylamide diethanolamine, tallowamide diethanolamine, lauramide monoisopropanolamine, tallowamide monoethanolamine, isostearamide diethanolamine, isostearamide monoethanolamine) and mixtures thereof. Still more preferably, the personal care composition of the present invention further comprises 0.01 to 80 wt % (more preferably, 1 to 50 wt %; still more preferably, 5 to 35 wt %, most preferably, 7.5 to 20 wt %) of a personal care cleaning surfactant; wherein the personal care composition is a body wash formulation and wherein the personal care cleaning surfactant is selected from the group consisting of alkyl polyglucosides (e.g., lauryl glucoside, coco-glucoside, decyl glucoside), glycinates (e.g., sodium cocoyl glycinate), betaines (e.g., alkyl betaines such as cetyl betaine and amido betaines such as cocamidopropyl betaine), taurates (e.g., sodium methyl cocoyl taurate), glutamates (e.g., sodium cocoyl glutamate), sarcosinates (e.g., sodium lauroyl sarcosinate), isethionates (e.g., sodium cocoyl isethionate, sodium lauroyl methyl isethionate), sulfoacetates (e.g., sodium lauryl sulfoacetate), alaninates (e.g., sodium cocoyl alaninate), amphoacetates (e.g., sodium cocoamphoacetate), sulfates (e.g., sodium lauryl ether sulfate (SLES)), sulfonates (e.g., sodium C₁₄₋₁₆ olefin sulfonate), succinates (e.g., disodium lauryl sulfosuccinate), fatty alkanolamides (e.g., cocamide monoethanolamine, cocamide diethanolamine, soyamide diethanolamine, lauramide diethanolamine, oleamide monoisopropanolamine, stearamide monoethanolamine, myristamide monoethanolamine, lauramide monoethanolamine, capramide diethanolamine, ricinoleamide diethanolamine, myristamide diethanolamine, stearamide diethanolamine, oleylamide diethanolamine, tallowamide diethanolamine, lauramide monoisopropanolamine, tallowamide monoethanolamine, isostearamide diethanolamine, isostearamide monoethanolamine) and mixtures thereof. Most preferably, the personal care composition of the present invention further comprises 0.01 to 80 wt % (more preferably, 1 to 50 wt %; still more preferably, 5 to 35 wt %, most preferably, 7.5 to 20 wt %) of a personal care cleaning surfactant; wherein the personal care composition is a body wash formulation and wherein the personal care cleaning surfactant comprises a mixture of a betaine (preferably, cocamidopropyl betaine), a sulfate (preferably, sodium lauryl ether sulfate (SLES)), and a fatty alkanolamide (preferably, cocamide monoethanolamine)

Preferably, the personal care composition further comprises a thickener. More preferably, the personal care composition further comprises a thickener, wherein the thickener is selected to increase the viscosity of the personal care composition, preferably without substantially modifying the other properties of the personal care composition. Preferably, the personal care composition further comprises a thickener, wherein the thickener is selected to increase the viscosity of the personal care composition, preferably without substantially modifying the other properties of the personal care composition and wherein the thickener accounts for 0 to 5.0 wt % (preferably, 0.1 to 5.0 wt %; more preferably, 0.2 to 2.5 wt %; most preferably, 0.5 to 2.0 wt %), based on weight of the personal care composition.

Preferably, the personal care composition of the present invention further comprises an antimicrobial agent/preservative. More preferably, the personal care composition of the present invention further comprises an antimicrobial/preservative, wherein the antimicrobial/preservative is selected from the group consisting of phenoxyethanol, benzoic acid, benzyl alcohol, sodium benzoate, DMDM hydantoin, 2-ethylhexyl glyceryl ether, isothiazolinone (e.g., methylchloroisothiazolinone, methylisothiazolinone) and mixtures thereof. Still more preferably, the personal care composition of the present invention, further comprises an antimicrobial/preservative, wherein the antimicrobial/preservative is a mixture of phenoxyethanol and an isothiazolinone (more preferably, wherein the antimicrobial/preservative is a mixture of phenoxyethanol and methylisothiazolinone).

Preferably, the personal care composition of the present invention optionally further comprises a pH adjusting agent. More preferably, the personal care composition of the present invention, further comprises a pH adjusting agent, wherein the personal care composition has a pH of 4 to 9 (preferably, 4.25 to 8; more preferably, 4.5 to 7; most preferably, 4.75 to 6).

Preferably, the pH adjusting agent is selected from the group consisting of at least one of citric acid, lactic acid, hydrochloric acid, aminoethyl propanediol, triethanolamine, monoethanolamine, sodium hydroxide, potassium hydroxide, amino-2-methyl-1-propanol. More preferably, the pH adjusting agent is selected from the group consisting of at least one of citric acid, lactic acid, sodium hydroxide, potassium hydroxide, triethanolamine, amino-2-methyl-1-propanol. Still more preferably, the pH adjusting agent includes is triethanolamine Most preferably, the pH adjusting agent is triethanolamine

Preferably, the method of depositing silicone of the present invention, comprises: selecting a personal care composition of the present invention and applying the personal care composition to at least one of a skin and hair. More preferably, the method of depositing silicone of the present invention, further comprises: rinsing the personal care composition from the at least one of skin and hair with a rinse water. Most preferably, the method of depositing silicone of the present invention, comprises: providing a personal care composition of the present invention; applying the personal care composition to hair; and rinsing the personal care composition from the hair; wherein the personal care composition is at least one of a shampoo and a conditioner (preferably, wherein the at least 15 mol % (more preferably, at least 17 mol %; most preferably, at least 20 mol %) of the silicone from the composition is deposited on the hair).

Some embodiments of the present invention will now be described in detail in the following Examples.

### Synthesis 51: Hydrophobically Modified Cellulose Ether Base Material

A 2,000 mL, four necked, round bottomed flask was charged with cellulose ether base material (140.4 g, CELLOSIZE™ QP-100MH hydroxyethyl cellulose available from The Dow Chemical Company), isopropyl alcohol (724.7 g) and deionized water (104.6 g). The flask was fitted with a nitrogen inlet connected to a 60 mL pressure equalizing addition funnel, rubber septum cap, a stirring paddle connected to an electric motor and a Claisen adaptor connected to a Friedrich condenser with a mineral oil bubbler outlet. The addition funnel was then charged with 1-bromododecane (48.2 g) in isopropyl alcohol (40 g). The stirring paddle was engaged and the head space in the flask was purged with a slow, steady flow of nitrogen (one bubble per second) for one hour to remove any entrained oxygen. Then a 50% aqueous sodium hydroxide solution (32.16 g) was added dropwise to the flask contents over two (2) minutes. The flask contents were left to stir for an hour following addition of the 50% sodium hydroxide solution. The 1-bromododecane in isopropyl alcohol solution in the addition funnel was then charged dropwise to the flask contents over three (3) minutes. The flask contents were left to stir for twenty (20) minutes following the addition of the 1-bromododecane in isopropyl alcohol. Then heat was applied to the contents of the flask using a heating mantle. The flask contents were allowed to reflux with continued stirring under nitrogen for four and a half (4.5) hours. The flask was then placed in an ice water bath while maintaining a positive nitrogen pressure in the flask to cool the flask contents. The flask contents were then neutralized via the addition thereto of glacial acetic acid (12.0 g) using a syringe. The flask contents were left to stir for ten (10) minutes under nitrogen. The flask contents were then vacuum filtered through a large fritted metal Buchner funnel to recover the hydrophobically modified cellulose ether base material. The recovered hydrophobically modified cellulose ether base material was then washed in the Buchner funnel by stirring in the funnel for five (5) minutes with the specified wash solvents followed by vacuum removal of the wash liquor: deionized water (216 g) in isopropyl alcohol (984 g); deionized water (120 g) in isopropyl alcohol (1,080 g); and then isopropyl alcohol (1,200 g) with 40% aqueous glyoxal (2.88 g) and acetic acid (0.88 g). The washed product hydrophobically modified cellulose ether base material was then briefly air dried and then dried overnight in vacuo at 50° C. The dried product hydrophobically modified cellulose ether base material was then sieved through a 30 mesh plate and obtained as an off-white solid (161.61 g) with a volatiles content of 15.44%, an ash content (as sodium acetate) of 11.58%.

### Synthesis S2-S14: Hydrophobically Modified Cellulose Ether Base Material

The product hydrophobically modified cellulose ether base material in each of Synthesis S2-S14 was prepared using the same process as described above for Synthesis S1, with appropriate changes in raw material charges to provide the hydrophobically modified cellulose ether base material with the hydrophobic substituent and degree of substitution noted in TABLE 1.

### Example 1: Modified Carbohydrate Polymer

A 500 mL, four necked, round bottomed flask was charged with the product hydrophobically modified cellulose ether base material prepared according to Synthesis S1 (26.08 g), isopropyl alcohol (168.52 g) and deionized water (29.45 g). The flask was then fitted with a nitrogen inlet connected to a 60 mL pressure equalizing addition funnel, rubber septum cap, a stirring paddle connected to an electric motor and a Claisen adaptor connected to a Friedrich condenser with a mineral oil bubbler outlet. The addition funnel was then charged with a 70 wt % aqueous solution of 2,3-epoxypropyltrimethylammonium chloride (25.85 g, available from SKW QUAB Chemicals, Inc. under the tradename QUAB® 151). The stirring paddle was engaged and the head space in the flask was purged with a slow, steady flow of nitrogen (one bubble per second) for one hour to remove any entrained oxygen. Then a 25% aqueous sodium hydroxide solution (2.42 g) was added dropwise to the flask contents over two (2) minutes. The flask contents were left to stir for an hour following addition of the 25% sodium hydroxide solution. The aqueous 2,3-epoxypropyltrimethylammonium chloride solution in the addition funnel was then charged dropwise to the flask contents over three (3) minutes. The flask contents were left to stir for twenty (20) minutes following the addition of the 2,3-epoxypropyltrimethylammonium chloride in isopropyl alcohol. Then the flask contents were heated at 55° C. for ninety (90) minutes. The flask was then placed in an ice water bath while maintaining a positive nitrogen pressure in the flask to cool the flask contents. The flask contents were then neutralized via the addition thereto of glacial acetic acid (2.5 g) using a syringe. The flask contents were left to stir for ten (10) minutes under nitrogen. The flask contents were then vacuum filtered through a large fritted metal Buchner funnel to recover the modified carbohydrate polymer. The polymer was then washed in the Buchner funnel by stirring in the funnel for five (5) minutes with the specified wash solvents followed by vacuum removal of the wash liquor: deionized water (36 g) in isopropyl alcohol (164 g); deionized water (20 g) in isopropyl alcohol (180 g); and then isopropyl alcohol (200 g) with 40% aqueous glyoxal (0.44 g) and acetic acid (0.15 g). The washed polymer was then briefly air dried and then dried overnight in vacuo at 50° C. The product polymer was then sieved through a 30 mesh plate and obtained as an off-white solid (27.25 g) with a volatiles content of 3.00%, an ash content (as sodium acetate) of 5.62%. The total Kjeldahl nitrogen, TKN, was determined to be 2.54 wt %. The 1% solution viscosity of the product polymer was measured at 6.31 sec⁻¹ using a TA Instruments DHR-3 rheometer equipped with a 40 mm, 2.0° stainless steel cone & plate sensor at 25° C. and was determined to be 17,721 mPa·s.

### Comparative Examples C1-C2 and Examples 2-12: Modified Carbohydrate Polymer

The product modified carbohydrate polymer in each of Comparative Examples C1-C2 and Examples 2-12 was prepared using the same process as described above for Example 1, with appropriate changes in raw material charges to provide the product modified carbohydrate polymer with the total Kjeldahl nitrogen, TKN, as reported in TABLE 1.

TABLE 1

| | | | | Modified Carbohydrate Polymer | | | |
|---|---|---|---|---|---|---|---|
| | Modified base material | | | | | | Viscosity |
| | | Hydrophobe | Hydrophobe | TKN | Volatiles | Ash | 6.31 sec⁻¹ |
| Ex. | Synthesis | Reagent | (mol %) | (wt %) | (wt %) | (wt %) | (mPa · s) |
| C1 | S13 | A | 0.024 | 2.06 | 5.67 | 3.81 | 1,559 |
| C2 | S14 | B | 0.024 | — | 4.09 | 4.58 | 11,664 |
| 1 | S1 | B | 0.036 | 2.54 | 3.00 | 5.62 | 17,721 |
| 2 | S2 | B | 0.014 | 2.36 | 4.45 | 2.92 | 1,981 |
| 3 | S3 | B | 0.014 | 1.92 | 8.89 | 2.62 | 3,150 |
| 4 | S4 | B | 0.024 | 2.51 | 4.86 | 2.71 | 6,303 |
| 5 | S5 | B | 0.024 | 2.20 | 4.01 | 4.89 | 6,322 |
| 6 | S6 | B | 0.024 | 2.22 | 4.13 | 2.32 | 10,664 |
| 7 | S7 | B | 0.023 | 2.80 | 5.56 | 4.95 | 13,850 |
| 8 | S8 | B | 0.036 | 1.71 | 4.30 | 5.09 | 14,298 |
| 9 | S9 | B | 0.023 | 1.88 | 4.63 | 4.14 | 14,669 |
| 10 | S10 | B | 0.023 | 2.37 | 5.05 | 4.53 | 16,206 |
| 11 | S11 | B | 0.014 | 3.19 | 4.01 | 4.89 | 1,597 |
| 12 | S12 | B | 0.036 | 2.22 | 4.89 | 4.87 | 21,375 |

A 1-bromododecane
B 1-bromooctane

### Comparative Examples CF1-CF5 and Examples F1-F12: Shampoo Formulations

A shampoo formulation was prepared in each of Comparative Examples CF1-CF5 and Examples F1-F12 using the generic shampoo formulation noted in TABLE 2.

TABLE 2

| Ingredient INCI name | wt % |
|---|---|
| Deionized water | q.s. 100 |
| 70 wt % aq. soln. Sodium Lauryl Ether Sulfate¹ | 12.9 |
| Polymer | 0.3 |
| Tetrasodium EDTA² | 0.2 |
| 45 wt % aq. soln. PEG-150 Pentaerythrityl Tetrastearate³ | 2.8 |
| Cocamide MEA⁴ | 1.0 |
| 30 wt % aq. soln. Cocamidopropyl Betaine⁵ | 6.0 |
| Phenoxyethanol and Methylisothiazolinone⁶ | 0.5 |
| 50 wt % aq. soln. Dimethiconol and TEA-dodecylbenzenesulfonate⁷ | 2.0 |

¹available from Stepan Company under tradename Steol ® CS-370
²available from The Dow Chemical Company under tradename Versene ™ 220
³available from Croda Inc. under tradename Crothix-PA-(MH)
⁴available from Croda Inc. under tradename Incromide CMEA
⁵available from Stepan Company under tradename Amphosol CA
⁶preservative available from The Dow Chemical Company under tradename Neolone ™ PE
⁷available from The Dow Chemical Company under tradename Dowsil ™ 1785 emulsion

The shampoo formulations was prepared in each of Comparative Examples CF1-CF5 and Examples F1-F12 using the following process: In a container, the 70 wt % aqueous solution of sodium lauryl sulfate (12.9 g) was dissolved in deionized water (70 g) and heated to 70° C. with constant stirring. The polymer noted in TABLE 3 was then added to the container with stirring. When the polymer was dissolved, the tetrasodium EDTS was then added to the container. Once the container contents reached 70° C., the 45 wt % aqueous solution of PEG-150 pentaerythrityl tetrastearate and the 30 wt % aqueous solution of cocamide MEA were added to the container. Then the 30 wt % solution of cocamidopropyl betaine was added to the container. The contents of the container were then allowed to cool. Once at room temperature, the phenoxyethanol and methylisothiazolinone preservative and the 50 wt % solids aqueous emulsion of dimethiconol and TEA-dodecylbenzenesulfonate were added to the container. The final pH of the product shampoo formulation was then adjusted to a pH of 5 using sodium hydroxide or citric acid as necessary and sufficient water was added to adjust the total formulation weight to 100 g.

TABLE 3

| Shampoo Formulation | Polymer |
|---|---|
| Comparative Example CF1 | Synthesis S13 |
| Comparative Example CF2 | Synthesis S14 |
| Comparative Example CF3 | Polyquaternium-10¹ |
| Comparative Example CF4 | Polyquaternium-10² |
| Comparative Example CF5 | — |
| Example F1 | Synthesis S1 |
| Example F2 | Synthesis S2 |
| Example F3 | Synthesis S3 |
| Example F4 | Synthesis S4 |
| Example F5 | Synthesis S5 |
| Example F6 | Synthesis S6 |
| Example F7 | Synthesis S7 |
| Example F8 | Synthesis S8 |
| Example F9 | Synthesis S9 |
| Example F10 | Synthesis S10 |
| Example F11 | Synthesis S11 |
| Example F12 | Synthesis S12 |

¹commercially available from The Dow Chemical Company under tradename UCARE ™ Polymer JR-30M
²commercially available from The Dow Chemical Company under tradename UCARE ™ Polymer KG-30M

### Silicone Deposition Analysis

The silicone deposition on hair from the shampoo formulations prepared according to Comparative Examples CF1-CF5 and Examples F1-F12 was quantified using X-ray photoelectron spectroscopy (XPS), which gives a quantitative elemental and chemical state information from the top 10 nm of the hair sample.

Hair tresses (6 g, European 8-hour bleached available from International Hair Importers) were initially washed in a 9 wt % sodium laureth sulfate (SLES) solution and rinsed with water flowing at 0.4 L/min for 30 seconds. Following the initial wash step, the hair tresses were then washed with a shampoo formulation of Comparative Example CF1-CF5 and Example F1-F12 by applying 0.6 g of the shampoo formulation to the hair tress and massaging in for 30 seconds on each side and then rinsing with water flowing at 0.4 L/min for 30 seconds on each side. The hair tresses were then evaluated using XPS. The XPS data were acquired from four areas per tress across 1 cm². The instrument parameters used are provided in TABLE 4. The mol % of the silicon from the shampoo formulation deposited on the hair is reported in TABLE 5.

TABLE 4

| | |
|---|---|
| Instrument | K-Alpha X-ray photoelectron spectroscopy (XPS) |
| X-ray source: | Monochromatic Aluminum K-alpha |
| Analyzer Pass | 200 eV (survey spectra) |
| Energy | 20 eV (high resolution spectra) |
| Take-Off Angle | 90° |
| Auto height | on |
| Analysis Area | 400 μm oval |
| Flood Gun | on |

TABLE 5

| | Si deposition |
|---|---|
| Shampoo Formulation | (mol %) |
| Comparative Example CF1 | 10.7 |
| Comparative Example CF2 | 0.5 |
| Comparative Example CF3 | 12.3 |
| Comparative Example CF4 | 11.4 |
| Comparative Example CF5 | 0.4 |
| Example F1 | 15.1 |
| Example F2 | 18.6 |
| Example F3 | 21.2 |
| Example F4 | 17.1 |
| Example F5 | 18.0 |
| Example F6 | 17.4 |
| Example F7 | 15.9 |
| Example F8 | 16.0 |
| Example F9 | 19.0 |
| Example F10 | 17.4 |
| Example F11 | 16.3 |
| Example F12 | 18.5 |

We claim:

1. A personal care composition, comprising:
a cosmetically acceptable carrier;
a cosmetically acceptable silicone;
a modified carbohydrate polymer; wherein the modified carbohydrate polymer comprises a cellulose ether base material functionalized with (i) quaternary ammonium groups, wherein the quaternary ammonium groups and wherein the modified carbohydrate polymer has a Kjeldahl nitrogen content corrected for ash and volatiles, TKN, of 1.0 to 4.0 wt %; and (ii) hydrophobic substituents, wherein the hydrophobic substituents comprise an alkyl group having an average of 10 to 20 carbon atoms; wherein the modified carbohydrate polymer comprises >0.005 to <0.05 moles of the hydrophobic substituents per mole of the cellulose ether base material; and wherein the hydrophobic groups are randomly distributed across the backbone of the cellulose ether base material; and wherein the cellulose ether base material has a weight average molecular weight, Mw, of >200,000 Daltons.

2. The personal care composition of claim 1, wherein the personal care composition is selected from the group consisting of a shampoo, a conditioning shampoo, a leave on hair conditioner and a rinse off hair conditioner.

3. The personal care composition of claim 2, wherein the personal care composition is selected from the group consisting of a shampoo and a conditioning shampoo.

4. The personal care composition of claim 3, wherein the hydrophobic substituents are bonded to the cellulose ether base material through an ether linkage or an ether linkage and a 2-hydroxypropyl group.

5. The personal care formulation of claim 3, further comprising a personal care cleansing surfactant.

6. The personal care formulation of claim 3, further comprising a chelating agent.

7. The personal care formulation of claim 3, further comprising a thickener.

8. The personal care formulation of claim 3, further comprising
a surfactant, wherein the surfactant comprises a mixture of sodium lauryl ether sulfate, cocamide monoethanolamine and cocamidopropyl betaine;
a chelating agent, wherein the chelating agent comprises tetrasodium ethylenediaminetetraacetic acid; and
a rheology modifier, wherein the thickener comprises PEG-150 pentaerythrityl tetrastearate.

9. The personal care formulation of claim 8, further comprising a preservative.

10. A method of depositing silicone, comprising:
selecting a personal care composition according to claim 1;
applying the personal care composition to at least one of a skin and hair.
